(19) United States
(12) Patent Application Publication
(10) Pub. No.: US 2019/0321132 A1
(43) Pub. Date: Oct. 24, 2019

(54) AUTOMATIC IDENTIFICATION TECHNOLOGIES IN SURGICAL IMPLANTS ...

(71) Applicant: Mobile Workforce, Inc., Port Orchard, WA (US)

(72) Inventors: David WEIR, Port Orchard, WA (US); John NEERGAARD, Port Orchard, WA (US); Stewart A. SKOMRA, Lisbon, OH (US); Greg BOOTH, Port Orchard, WA (US); John SUTTON, Austin, TX (US)

(21) Appl. No.: 16/392,566

(22) Filed: Apr. 23, 2019

Related U.S. Application Data
(60) Provisional application No. 62/661,512, filed on Apr. 23, 2018.
(60) Provisional application No. 62/661,518, filed on Apr. 23, 2018.

(51) Int. Cl. A61B 90/98; A61B 90/96; A61B 50/33

(52) U.S. Cl. CPC A61B 90/98; A61B 2017/00526; A61B 50/33; A61B 90/96

(57) ABSTRACT

An article of manufacture such as surgical steel suspends an elongated antenna radiator above the surface of the steel. The steel surface acts as a ground plane that increases transmission/reception efficiency of the suspended radiator at UHF frequencies. Other embodiments support building reconfigurable surgical trays and the population of same with surgical instruments through a mixed or virtual reality human-machine interface to generate Centerline Gerber/Plot File data for Computer Numerical Control production and Human and/or Robotic Assembly of reconfigurable (or non-reconfigurable) Surgical Trays and the Human and/or Robotic population of Instruments in the Surgical Tray. Example applications include the use of the original Surgical Implant, Surgical Instrument, Surgical Tray, and other related Asset design and manufacturing databases including lot control for pre-populating a digital catalog/database that is used for both Human and Machine Surgical Set assembly and disassembly. Also, this same process may take as input high-resolution imagery and physical attribute capture of Assets to create a next-best-than original facsimile to the original design database and reconstitute a design database from this facsimile

### CROSS-REFERENCE TO RELATED APPLICATIONS

This application claims benefit of U.S. Provisional Application Nos. 62/661,512 and 62/661,518 each filed Apr. 23, 2018, incorporated herein by reference.

### STATEMENT REGARDING FEDERALLY SPONSORED RESEARCH OR DEVELOPMENT

None.

### FIELD

The present technology relates to automatically detecting and tracking collections of assets including but not limited to, as one example, tool collections such as arrays of surgical instruments. Some aspects of the technology herein relate to computer vision or other sensing using for example artificial intelligence or other machine learning to inventory collections. Example applications include the use of the original Surgical Implant, Surgical Instrument, Surgical Tray, and other related Asset design and manufacturing databases including lot control for pre-populating a digital catalog/database that is used for both Human and Machine Surgical Set assembly and disassembly. Also, this same process may take as input high-resolution imagery and physical attribute capture of Assets to create a next-best-than original facsimile to the original design database and reconstitute a design database from this facsimile Other aspects relate to electronic or digital tagging including but not limited to RFID and NFC tagging, and more particularly to techniques for assembling such tags with articles such as for example medical devices and surgical instruments to make the articles inseparable from the tags without interfering with the function of the tags or the instruments. Some aspects relate to attaching RFID Tags to Assets through additive manufacturing processes such as building tags directly on or within the surface of an underlying Asset to which the RFID Tag is intended to be associated.

### BACKGROUND

The medical device industry is valued at over $250B annually. Up to 8% of such medical devices are lost or unaccounted for every year. Often this shrink is due to poor inventory control where expensive devices simply go missing. This adds up to over $3B in losses every year.

1D and 2D bar codes have been used in the past to track devices. Some such bar codes comprise stickers that adhere to an external surface of a device. In some cases, bar codes have been etched into the surface of devices such as surgical instruments so the bar codes can survive sterilizing in an autoclave.

Electronically readable digital tags such as RFID and NFC tags are commonly used to associate readable (and sometimes also writable) digital information with an asset such as an article of manufacture. Generally speaking, such tags comprise a miniature transponder coupled to a non-transitory memory device storing at least an identifier. When the device is read by sending it radio frequency (electromagnetic) signals of a particular frequency, the transponder responds by wireless (non-contact) transmission of digital information the memory device stores. In the simplest example, the digital information received back from the transponder can name or identify the article with a unique identifier that distinguishes the article from all others.

One example deployment of such technology is in the health care industry. For example, work has been done in the past to deploy RFID technology in hospital environments. See for example US20140125482 and US20170143431, each incorporated herein by reference. In hospitals, surgical tools such as scalpels, scissors, clamps, and retractors are needed for surgeries daily. This equipment needs to always be on hand, clean, disinfected, and ready to use. Not having the right surgical equipment on hand may be the least dangerous situation caused by not tracking these tools. Studies show that surgical instruments in some hospitals are found to carry bacteria from previous use, either due to not being sterilized or not being sterilized properly. Not only could tracking these items with RFID tags ensure that each tool is sterilized prior to use—a properly implemented system may be able to shed light on sterilization methods for individual tools. See Smiley et al, “7 Things You Can Track in Hospitals Using RFID”, RFID Insider 8/21/17, retrieved at https://blog.atlasfidstore.com/7-things-can-track-hospitals-using-rfid; “RFID Surgical Instrument Tracking Systems Prevent RSI” (Smyrna Technology Jul. 1, 2016), retrieved at https://www.syrmatech.com/rfid-surgical/

On-metal RFID tags embedded in or applied on surgical equipment can allow the surgical equipment to be tracked for inventory purposes and ensure the equipment went through the disinfecting process of the autoclave. A few types of autoclaves exist that use different sterilization methods; by tracking each tool individually, hospitals can ensure that all tools went through the proper autoclave specified by the manufacturer. Not all RFID tags can survive the autoclave process so tags must be chosen to ensure all equipment is properly cleaned and sterile. See Smiley et al.; see also Bendavid et al, Managing Surgical Instruments, RFID Journal (05/22/16), incorporated herein by reference.

Meanwhile, a fundamental challenge with RFID as a commercial offering is the total cost of ownership associated with the infrastructure required to read (and sometimes read/write from/to) the RFID Tags. The cost of readers/interrogators are continually decreasing. For example, many smart phones, tablets and other consumer and industry electronics devices now incorporate NFC readers. However, fixed interrogator systems in the context of a hospital environment such as an operating room or a sterilization station can still be quite expensive.

Some have proposed attaching RFID tags to surgical instruments and using an RFID interrogator to automatically interrogate the tags on the surgical instruments. Such interrogator could automatically provide information concerning which (and how many) surgical instruments are in a tray, and track the usage of each surgical instrument (e.g., which surgeries was the instrument previously used for, when was it purchased, when was the last time it was sterilized, etc.) Such interrogation could for example assure a surgeon that all of the instruments she requires to perform a surgery are present in the tray and have all be properly sterilized.

FIGS. 2A and 2B show an example prior approach of how some have incorporated an RFID tag 12 into a surgical instrument 10. In the example shown, an RFID tag 12 such as an “integrated circuit” is purchased as a unit and adhered to a surgical steel surface 14 of steel surgical instrument 10 using a layer of adhesive 18. An additional encapsulation layer 20 is then applied over the RFID tag 12 to establish a bond that is impervious to liquid or steam used to sterilize the surgical instrument 16 in autoclave A or the like. The encapsulation layer 20 is preferably substantially transparent to radio frequency transmissivity, and can be sterilized along with the instrument 10 so the assembly is incapable of harboring pathogens. In the example shown, there is no electrical connectivity between the RFID tag 12 and the steel material comprising the instrument 10. Rather, the RFID tag 12 in this prior approach is self-contained and is manufactured separately to operate independently of any external structures.

To construct such an instrument 10 as shown in FIG. 1, the surgical instrument manufacturer or end user typically purchases manufactured RFID integrated circuits 12 from another manufacturer or manufactures them separately. The surgical instrument manufacturer procures the adhesive 18 and encapsulation agent 20 and performs the assembly/manufacturing shown in FIGS. 2A and 2B.

A problem with this prior approach is that the different components (tag 12 and steel 10) have different heat expansion coefficients. Accordingly, repeated sterilization operations in an autoclave A can cause the adhesive layer 18 and/or adhesive encapsulation 20 to begin to crack. Cracks in such material could harbor pathogens such as bacteria that could cause infection when the instrument 10 is used for surgery. Therefore, the RFID tag 12 application may substantially shorten the useful life of the surgical instrument 10.

Despite such prior work, there is still the problem of how to attach, in a permanent (non-separable way), a digital tag to the article with which it is associated. It is generally known to embed RFID tags into some kinds of articles, including for example running shoes. However, further improvements are possible and desirable.

For example, the core focus of most health care providers is patient care efficacy. However, they are often distracted by:
Bundled payments driving to ASCs and out-patient
ASCs expanding into more complex procedures
˜50% SPD trays processed are consignment & manufacturers
Providers out of space
Central sterile processing high staff turnover Central sterile Processing increasing regulatory scrutiny.

Similarly, manufacturers often have a core focus of Product Innovation Value but are often distracted by:
Decreasing surgeon alignment
Decreasing selling prices
Expanding number of provider locations to support
Instrumentation becoming more complex, & difficult to clean & sterilize
IFU—Instructions for Use difficult for provider to locate and not routinely updated
Increasing distribution, costs and regulations.

Additionally, some applications could benefit from solutions other than RFID/NFC tags. For example, other technologies including magnetic auto identification, GPS (global positioning system) and/or LPS (local positioning system) and/or mixed-reality enhanced systems may be possible.

### BRIEF DESCRIPTION OF THE DRAWINGS

The following detailed description of exemplary non-limiting illustrative embodiments is to be read in conjunction with the drawings, of which:

FIG. 1A shows an example prior art surgical tray.

FIG. 1B shows an example prior art usage of RFID technology with surgical instruments.

FIGS. 2A and 2B show a prior approach for affixing an RFID tag to a surgical instrument.

FIG. 3 shows an example non-limiting embodiment of the technology herein that builds up an RFID tag structure on the surface of a surgical instrument.

FIG. 4 shows an example non-limiting RFID integrated circuit 34.

FIGS. 5A and 5B show an example hospital use case.

FIG. 5C shows an example process.

FIG. 6 shows an example automated detection system.

FIG. 7 shows an example non-limiting process.

FIGS. 8A-8E illustrate, in a non-limiting way, the process of FIG. 7.

### DETAILED DESCRIPTION OF EXAMPLE NON-LIMITING EMBODIMENTS

Example Use Case—Surgical Instruments

While application of the technology herein is not limited to surgical instruments, the following provides a non-limiting discussion of one example application of the technology in the context of surgical instruments and trays.

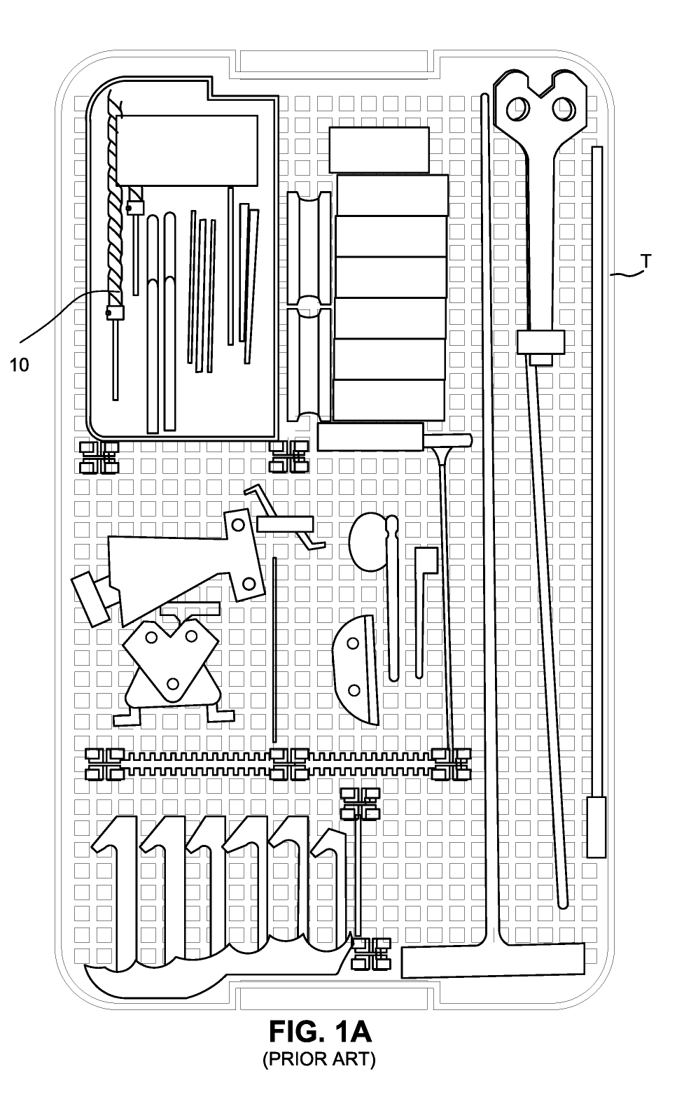

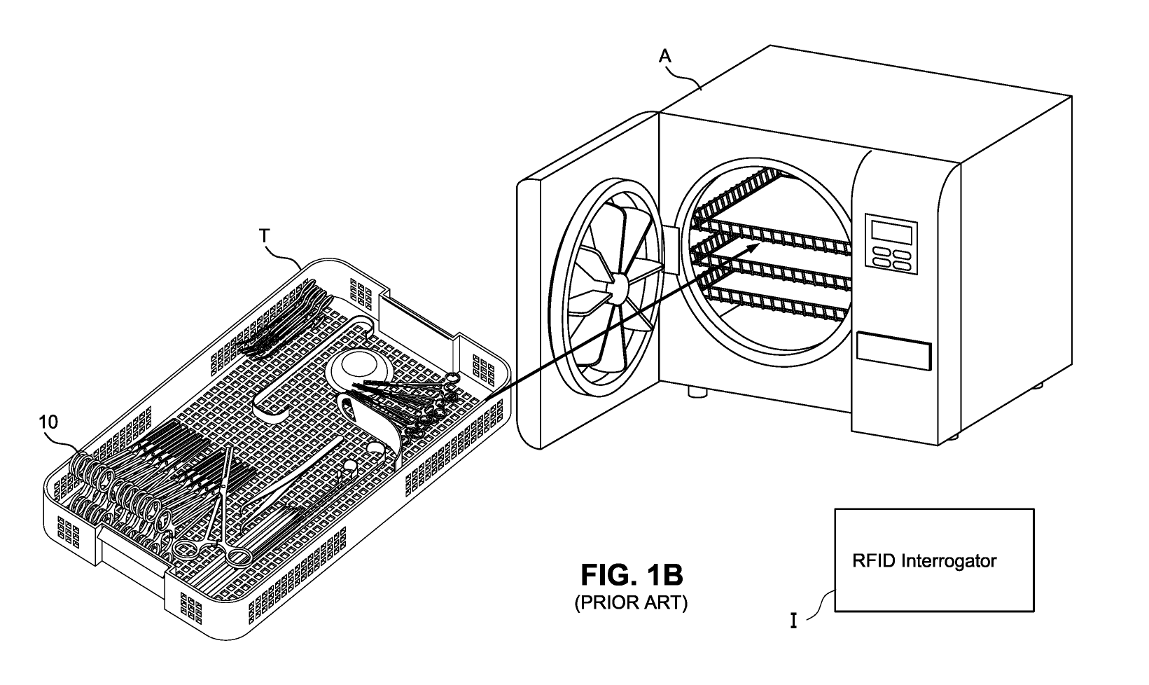

FIGS. 1A & 1B show a contemplated use case in the context of surgical instruments. Trays T are made from a sterilizable material such as surgical steel, and are configured to hold various steel surgical instruments 10 such as scalpels, scissors, clamps, drills, and retractors. Different trays T can be configured for different types of surgeries. For example, the tray T used for thoracic surgery may be different from the tray used for spinal surgery and may contain different instruments and in some cases different implants or other devices to be used in the surgery. Some surgeries may require multiple trays T. One or a number of trays T containing appropriate items 10 can be considered a full or partial surgery kit containing whatever a surgeon needs to perform a particular procedure. Thus, different types of surgeries require different kits, and different surgeons performing the same procedure may require different kits depending on individual surgeon preferences.

To prevent infection, these trays T and the surgical instruments or other items 10 they contain must be sterilized in an autoclave A before each use. Within the autoclave A, the trays T containing surgical instruments and other items 10 are exposed to steam and heat which does no damage to the steel or other materials out of which the surgical instruments and other items are made, but can damage other components (especially electronic components) attached to the surgical instruments. Autoclaves A typically use high pressure (15 psi) steam at temperatures between 250 and 273 degrees F., so components will have to easily withstand that. Depending on the type of trays and type of autoclave, the trays T are sometimes stacked before being placed in the autoclave A.

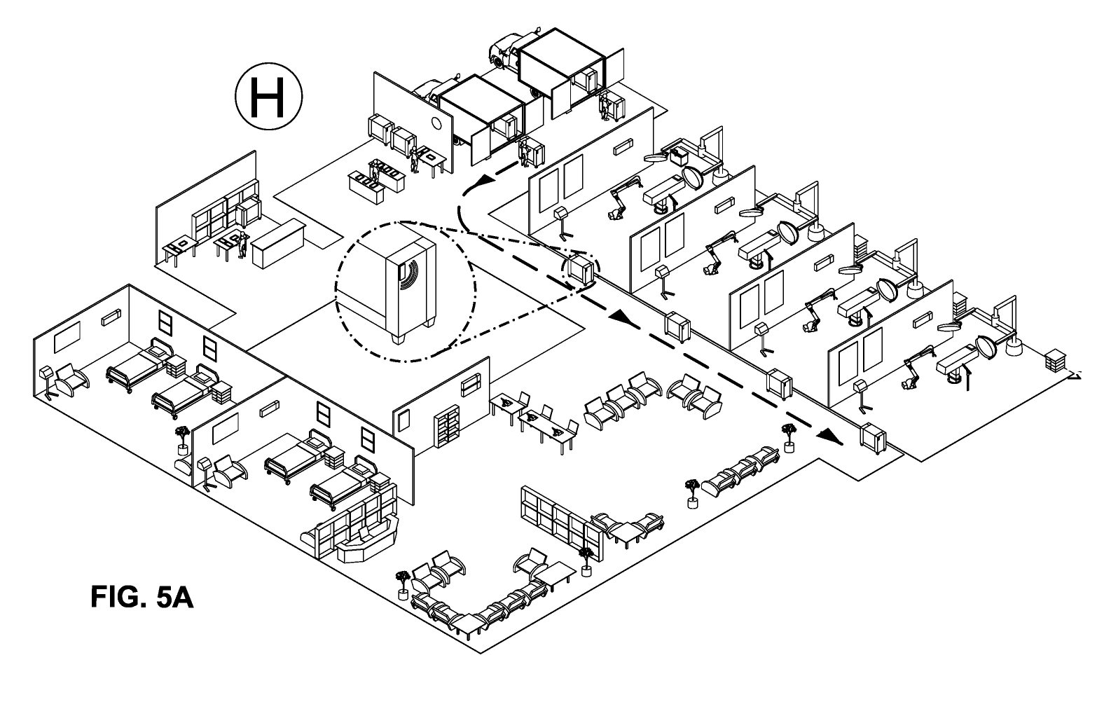

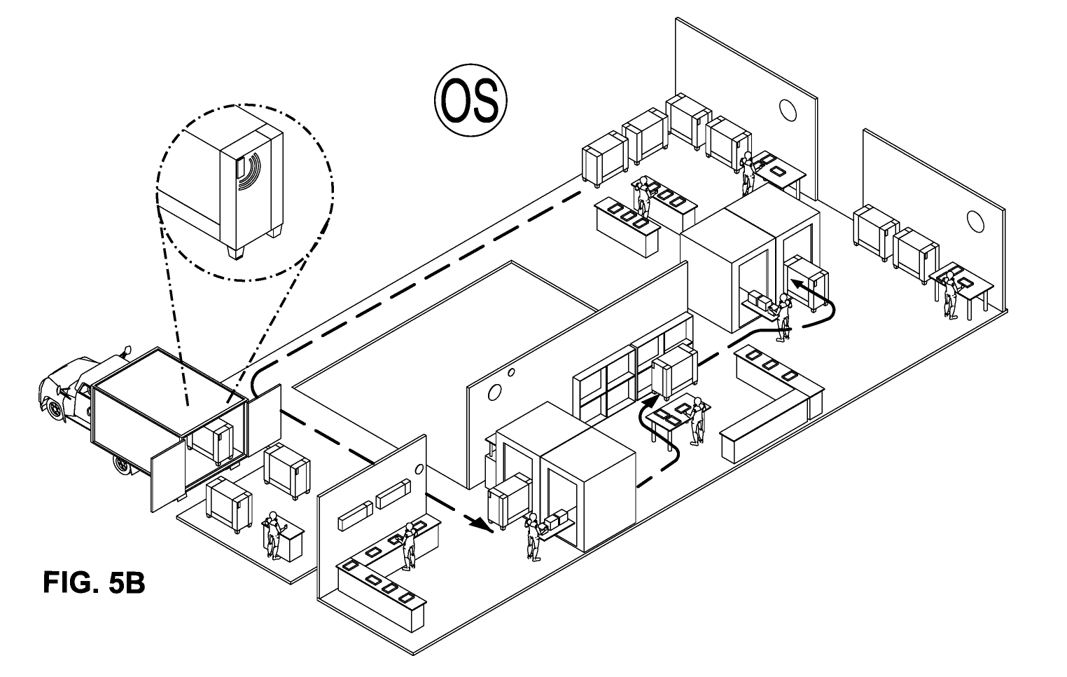

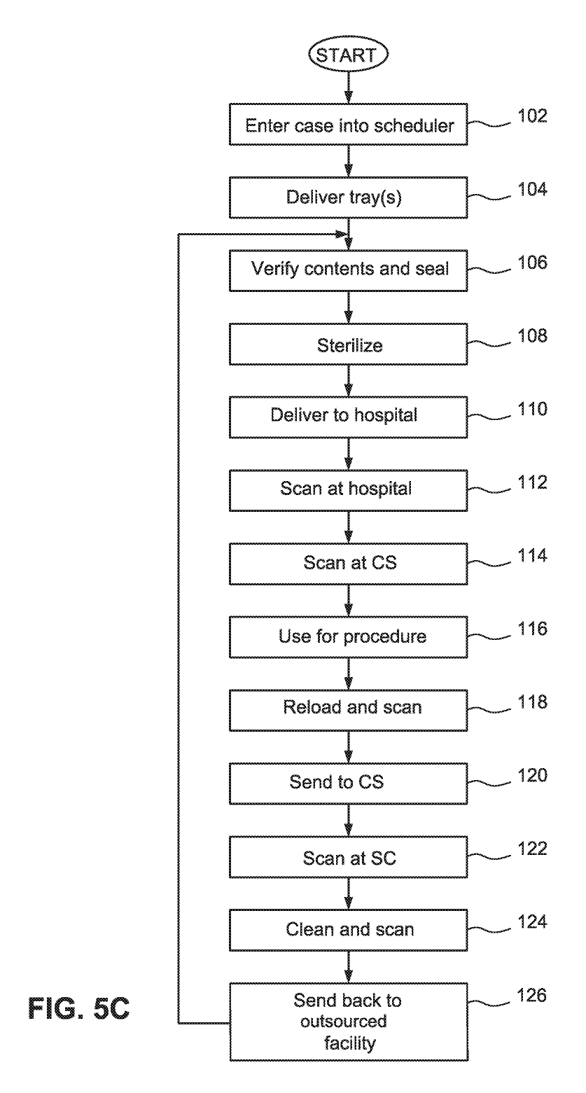

In some cases, the trays T are supplied from an outsourced sterilization facility separate from the hospital or other health care facility. As shown in FIGS. 5A, 5B and 5C, case information can be entered to the case scheduler (102), which emails or otherwise communicates copies of the case data to involved parties. A third party vendor will then create surgical kits based on the surgeon and/or surgical procedure, and deliver populated trays to the outsourced sterilization facility using a return tagged container (104). A container tag is associated with each tagged tray and tray contents are verified before sealing (106). The trays are sterilized, and autoclave number and cycle are linked to the sealed container tag to certify load (108). The container is then loaded for delivery from the outsourced sterilization facility to the hospital to begin the cycle (110).

In the example non-limiting context of a hospital setting, a driver/hospital scans container arrival and transfers custody to the hospital (112). The container is then scanned by central sterilization (CS) within the hospital at operating room delivery (114). The container is then used for a surgical or other procedure (116).

Once the surgical or other procedure is completed, all trays are reloaded with the now non-sterile surgical instruments, and the reloaded container is scanned (118) and sent to central sterilization (120). At central sterilization, the cabinet is opened and its contents are verified by central sterilization scanning (122). Central sterilization adds enzymatic cleaner, seals the container, and scans it for return (124). The hospital notifies the outsourced sterilization for pickup (126). The hospital/driver scans the cabinet for transferring custody to the driver at time of pickup and return to the outsourced sterilization facility.

As one can see from the FIG. 5C process, there are numerous times when scanning the contents of the tray T may be required: before and after delivery, before and after use in surgery, etc. Automated scanning of the contents of tray 10 would be highly desirable. The example non-limiting embodiments provide multiple techniques and options for providing such scanning to provide an end-to-end system custody chain.

Example non-limiting embodiments further provide on-vehicle tracking to monitor the following while equipment is being transported from one location to another:
Cabinet & Set Status
Intrusion Detection
Geozone Entry-Exit
Vehicle Monitoring
Driver Safety.

Example embodiments provide a patient-empowered, physician-directed, provider-optimized effective and efficient healthcare ecosystem delivering quality of service, quality of care, and quality of life for all stakeholders. Example non-limiting technology herein drives continuous quality and cost performance improvement by planning-scheduling-executing the complete perioperative supply chain on behalf of healthcare providers and medical device manufacturers serving physician-patient-procedure preferences. Example embodiments also provide visibility of Inventory at all points from every perspective; documented, Seamless Order Entry-Staging-Delivery; and Ability to Manage Change.

Further example non-limiting embodiments provide:
“One-Logical View”—with role-based access enabling a single system view of multiple disparate legacy systems integrated with the surgical case management software automation.
Single software application
Interfacing with provider & manufacturer's systems
Continuous technology development
Providing a means for assets to communicate with each other.
Logistics support
Case Management & Kit Processing
Sterilization
Real-Time Execution Access:
   Integrated to surgical schedule applications
   Quotes
   Sales orders
   Inventories
   Contract price validation
   Invoicing
Role-based access Example: Support hospital and manufacture representative inventories by lot number
No Architecture or Systemic Limits
Integration to both Hospital & Manufacturer systems
Interfaces for Proprietary & Legacy System
Transform ‘Islands-of-Automation’ in stagnant ‘Data-Lakes’ into Streams of Value-Generation
Case-Driven & Case-Driving End-to-End Supply-Chain Management
Delivery of loaner and provider sterile sets saving time-space-capital investment
Methodical incremental automation: Crawl-Walk-Run
Reducing costs while concurrently improving performance-quality-consistency
Structured Methodology:
   As-Is Assessment
   Transition Planning
   Execute Transition
   Operate, Stabilize & Support
   After-Action Review & Repeat
Improved Efficacy Achieved While Maintaining & Building Your Extended Enterprise Momentum

RFID/NFC Scanning

One example non-limiting technology herein assembles an RFID transponder directly on the surface of the surgical steel 10. See FIG. 3. In the non-limiting embodiment shown, the RFID or other electronic non-contact transponder 50 includes electronic components such as a charging circuit, a resonant RF circuit, a non-transitory memory device storing (or able to store) digital information, and in some embodiments, a processor or a processing circuit.

Transponder 50 in some non-limiting embodiments is designed and structured to be completed by being further assembled with/to other components external of the RFID transponder integrated circuit 36. In the example shown, the transponder assembly includes a ground standoff 32 and an RFID antenna 34 in addition to RFID integrated circuit 36. The ground standoff 32 is placed in electrical and physical contact with the surgical steel surface 14, establishing a galvanic (current-carrying) connection between the surgical steel surface 14 and the ground standoff. This connection converts the steel surgical instrument 10 into a portion of the transponder 50—namely a ground plane for an additional, parallelly-oriented RFID antenna radiator 34 that is suspended above the surgical steel surface 14 by an adhesive encapsulation layer 38. The RFID antenna 34 may in some example implementations comprise an elongated conductor such as copper, aluminum or other conductor that radiates and receives radio frequency signals of particular frequencies. As is well known by those skilled in the art, the length of the RFID antenna may be dimensioned to tune a particular transmit/receive frequency or it may function as a “long wire” to receive and transmit over a wide range or band of RF frequencies.

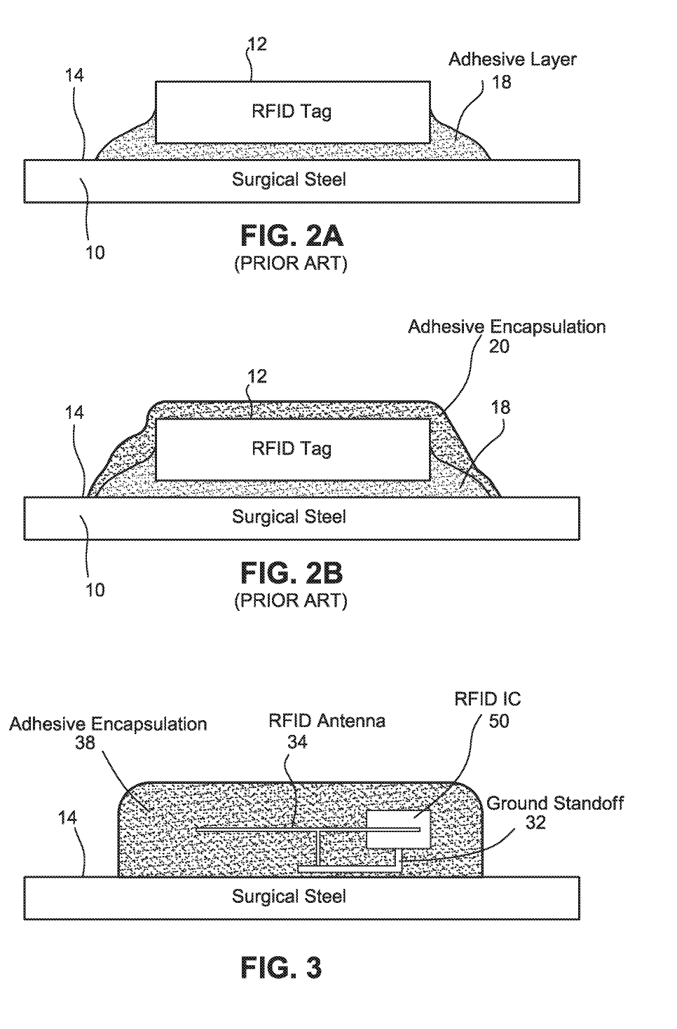

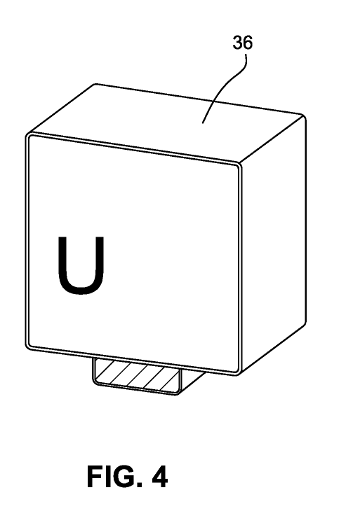

The VSWR and sensitivity and efficiency of the RFID antenna 34 is improved substantially by suspending the antenna above the ground plane provided by the surgical steel surface 14 acting as a ground plane. Because the RFID antenna 34 is spaced above the surgical steel surface 14 (in some embodiments by a particular spacing and separated by an adhesive encapsulation layer 38 having appropriate dielectric characteristics, all of which relate to the frequency range of interest), the RFID antenna 34 (having a length which may comprise a quarter-wave, half-wave or other radio frequency related dimension) provides a more efficient radiator and receiver of radio frequencies of interest. Furthermore, because the RFID antenna 34 is elongated along the length of the surgical instrument 10, it can be designed to operate efficiently at lower or different radio frequencies than the short wavelengths that conventional self-contained RFID tags of the type shown in FIGS. 2A, 2B rely on. For example, the elongated RFID antenna 34 may be dimensioned to operate efficiently in some portion of the 300 MHz-1 GHz radio frequency range. In some implementations, this tiny assembly (example shown in FIG. 4) has a 1 meter/3.3 ft read range at such UHF frequencies—and also may include a selective response/anti-collision protocol.

Known suppliers of the IC 36, Antenna 34, and any of the Ground and Standoff elements 32 that are contained within tags such as the HID RFID Brick Ceramic Tag Part#698930 (EU 869 MHz) and Part#698931 (US 915 MHz) may be employed. By, in some non-limiting embodiments, removing (i.e., omitting) the ceramic within which the parts are encased and then having a encapsulation material 38 and process defined, we obtain both a higher durability solution, simpler assembly, and—with the possibility of the Surgical Steel surface 14 serving as a ground plane—a potentially better read-range distance performance solution.

One example non-limiting embodiment builds the RFID Tag 50 directly on a Surgical Instrument 10 using the encapsulation material 38—with the proper dielectric constant as the:
1. RFID Tag Binder/Potting Material
2. Adhesive
3. Sealant

This approach solves the problems of Gen1 Passive RFID Tags of FIGS. 2A, 2B while also having the potential to be much higher RF performance, and also a faster application than the Gen1 multi-step process of FIGS. 2A, 2B. Such technology can allow scanning without touching the trays and cubes, individual medical device identification, complete trays and cabinet configuration, reading through water and metal (even inside of autoclave) and a combination of active and passive tags.

Dielectric properties and IC—Integrated Circuit assembly. The example non-limiting embodiment thus provides the concept of “fabricating” the Transponder 50 directly on the underlying Asset and using the Asset as Ground-Plane.

Characteristics of the adhesive and sealant determine the temperature ranges to work with in accomplishing the task(s). The temperature range within which the components will survive (i.e. IC, Antenna, and IC-to-Antenna bond) will limit the selection of materials and fabrication processes to withstand high pressure (15 psi) steam at temperatures between 250 and 273 degrees F.

Existing sealants having suitable characteristics (e.g., dielectric constant) may serve as the adhesive potting/bonding-sealant. High dielectric constant (eᵣ) low loss tangent (tan_{δ}, AKA Dissipation Factor (D)) are the electromagnetic requirements for making antennas 34 this small. High dielectric constant leads to smaller antennas and low loss tangent leads to less energy dissipation in the material. An eᵣ of 200 wouldn't be out of the question, but typically with higher tan_{δ} gets higher. The main requirement for this application would be miniaturization, so eᵣ is of primary interest. Of course heat resistance for this is also important as well as durability. These instruments will likely be horribly abused during handling, sterilization, and repackaging so the structures must be suitably rugged to withstand such treatment.

A major antenna optimization challenge for this application is to decouple the antenna from the vastly varying instrument geometries. Since the antenna is so close to the instrument, there will be very tight coupling and therefore the instrument will influence the antenna performance and thus the read range. Since the geometry of the instruments vary so vastly, it is not practical to design the antenna to take advantage of the instrument, i.e., basically coupling the IC to the instrument is the goal, but it is desirable to properly couple the IC to all the different potential geometries and still have good performance. In one example embodiment, the RFID IC 36 is attached to the instrument 10 and the antenna within the RFID IC becomes merely a coupling mechanism to couple the RFID IC to an external antenna. These will then be read by an interrogator I, i.e., range, time within range, multiple read requirement, and the like.

An RFID IC—Integrated Circuit Chip producer (such as TI—Texas Instruments) can, in other embodiments, sell a “bare” IC die 36 to companies who want to build RFID Tags. Such a die may be designed so that the actual IC Die can get very hot and still survive. The same holds for the Antenna 34 and the Antenna-to-IC bond.

The resulting instrument may in some applications be useful or used for a very short-range application. A relatively slow interrogation line using one or more interrogators I can be established that would produce good reads for all instruments/tags 10 passing by it. It would be possible for a reader to read multiple times and discard duplicates.

Example Non-Limiting Manufacturing Process

The example non-limiting embodiments shown in FIG. 3 should be able to achieve equal or better performance than the HID Model 60 and Model 70 RFID Brick Tags. See data sheet incorporated by reference entitled “Miniature Tags Mount Or Embed Discreetly To Tools, Equipment, Medical Instruments And More”, hidglobal.com (2017).

With the ability to more-or-less “custom-build” tags on-the-fly as FIG. 3 shows, the tag size could be variable. However, for ease of manufacturability, it may be best to have standard sizes around to make it possible to optimize tradeoffs of individual dimensions, read range, time to assemble and time to cure/dry.

The tag transponder 50 should desirably be bonded to the instrument well enough to withstand the heat and pressure of the autoclave A and what will be some abusive handling along the way. However, the tag 50 is typically not at risk to be knocked off by larger instruments or in typical handling and use. A cohesive bond may be preferably over an adhesive bond. The final assembly may have environmental characteristics like the Surgical Steel surface 14 itself.

For picking up when applied, a relatively flat surface will likely be helpful for easy robotic pickup (suction cup or the like). A SCARA-like robot with a suction part holder may be used to retrieve the RFID Tag “Innards” 36, place them at the mounting point on the steel surface 14 of the Article being tagged, and then then a base of the encapsulation material (i.e., “a footer”) 38 poured around the assembly while the suction tag holder is removed—the encapsulation material would then continue to be placed/deposited/3D-printed around and in the RFID Tag “Innards” 36 until it is completely covered. The extrusion of adhesive encapsulation 38 might be similar for example to Daily Queen soft-serve ice cream dispensing.

The encapsulant 38 should be stable to steam sterilization and not block the wireless read or other communications. To optimally design the ultimate encapsulation, it is necessary to know the max temperature the RFID transponder 50 can withstand before being damaged, and what properties of the encapsulant interfere with the wireless read. For example, a metal might be used as the encapsulant if it does not block the signal. A certain range of dielectric constants can be met to assure a proper read. No optical transparency is required, but RF transparency is related to the loss tangent mentioned below.

The wire bonding of the RFID integrated circuit 36 wafer to the antenna 34 will in some embodiments be the most sensitive to heat. The temperature limit for wire bonds should be observed. The maximum temperature before damage to wafers vary, from one manufacturer to another. One potential value is 173 F. Others may be over 200 F.

Certain dielectric properties would be helpful to achieve a very small size resonant antenna 34. Antenna designs may use materials with dielectric constant (eᵣ) of 200. Although materials with this high a dielectric constant do provide significant miniaturization of the antenna element, they tend to be lossy, so there will likely be a tradeoff between dielectric constant and dissipation factor (DDF) or loss tangent (tan_{δ}) to find the right balance that leads to a reasonable antenna that is highly miniaturized and that radiates as efficiently as possible. The final product will have other considerations, but the rest of the antenna details should only minimally effect the rest of the design in some embodiments. A dielectric constant of around 90 seems to be a good median value to start with and as always when dealing with antennas loss tangent as low as possible.

In some embodiments, the transponder integrated circuit 36 electrically connects to surgical steel 14 and uses the surgical steel as an antenna/radiator as opposed to a ground plane.

Addressing the Challenge of Concurrent Reads

In some applications, interrogation may be individualized. In other applications, the marked assets might be just randomly piled on top or one another (see FIG. 1). For example, multiple instruments piled on top of one another in a sterilization tray T need to be able to perform multiple reads with tags near one another and haphazardly arranged—this is where the 1 Meter Range and an Anti-Collision protocol comes into play. Specifically, it is possible to use RFID IC 36 with an anti-collision protocol (e.g., random back off or the like) to allow all or many instruments to be queried or polled simultaneously and nevertheless receive useful reporting information from each individual instrument.

### Example Embodiment: Mobile Mesh Network

In another example non-limiting embodiment, the concept of a conveyor belt has been for the robotic assembly of the tags onto the surface of the instruments. That belt could only move as fast as the slowest assembly operation. For individual tag reading, the read rate could be extremely high compared to the speed of a conveyor belt. The RFID tag 50 technology may incorporate mesh network communications such as a 802.15.4 mobile mesh network to decrease or eliminate the need for expensive fixed infrastructure.

In particular, IEEE 802.15.4-based RSAE Labs Mist® Mobile Mesh Net (see rsaelabs.com website, incorporated herein by reference) can be used to solve the problems of and for RFID and IEEE 1902.1 RuBee. RSAE Labs Mist® Mobile Mesh Net solves the problem of the Infrastructure by equipping portable and mobile RFID readers with Mist® capabilities. In more detail, generally, RFID/NFC tags can be passive or active. For Active RFID tags, the IEEE 802.15.4 technology is continuing to plummet in cost and is likely to displace the UHF Active RFID offerings. For the Passive UHF RFID and for the IEEE 1902.1 RuBee Magnetic ID technology (which has potential to be passive, however, commercial offerings are active with+10 year battery life), by equipping Mist® nodes with Passive UHF RFID Tag Readers and/or with IEEE 902.1 RuBee readers, the Infrastructure is dynamically and securely assembled and reassembled along the constructs of a distributed mesh network. There is thus no need for the heavy total cost of ownership associated with dedicated, proprietary fixed RFID infrastructure.

In such embodiments in the context of FIG. 1, the various surgical instruments 10 would communicate with one another and each instrument would transmit its respective information as well as relay information transmitted by other surgical instruments within tray 10. An interrogator I would then operate merely as yet another node on the mesh network.

Some example embodiments provide a fully-self-contained wireless sensor node (=definition of a “Mote”) that becomes a permanent ‘digital payload and logging carrier’ that flows continuously through the postal and courier systems. The “Self Mailer” would be the device itself—it would have Auto-ID imprinted/etched on its outer surface and can optionally include, for ease of authentication with the Shipping party, a microphone+camera for audio and imagery capture. The audio and imagery would be part of the digital payload and would be used for mutual authentication of the shipper-to-mote and mote-to-oparcel/contents in addition to serving as meta data when combined with the encapsulated/embedded sensors within the Mote capturing “Data about the Shipment Data”. Such mote data can be the carrier of digital payloads that may be the entire shipment (i.e. simply bits, nibbles, and bytes) and also digital payloads that provide an authenticated trust bod to the tangible payload and continuously.

The digital payload may include components of a “distributed ledger” comprising for example both:
ASN—Advanced Shipping Notice and
Manifest.

In transit, the changes in the cargo itself—digital or otherwise—will be captured for modification of the Digital Manifest through Debit-Credit and the ASN-Manifest Reconciliation Process will be continuous and complete at the time of the Shipment Receipt.
By using Auto-ID enabled Mutual Authentication and Telematic Meta-Data, the reconciliation of Planned-versus-Actual Results will occur continuously and as a byproduct of the Custody Chain itself. An example embodiment would equip a container with a Fully-Operational Compute-Control-Communication Node (a.k.a. ‘Mote’=Wireless Sensor Node) equipped directly with Sensors or having access to Sensors and serving as a Depository/Repository for electronic record of the shipment's:

1. Advanced Shipping Notice (ASN)

2. Confirmed Physical Contents and their Condition at the point of the pre-trip Loaded Inventory

3. A continuous monitoring of Loaded Inventory Condition.

Such data goes beyond the type of RFID that is a limited subset of Auto-ID.

The implementation of the above capabilities then results in the ASN & Manifest Reconciliation being performed continuously in-transit. This “Plan versus Actual Continuous Reconciliation” at all points, everywhere—between facilities, within cargo transport itself, within facilities, will apply to EVERYTHING especially when we look to a future of Fully Autonomous, Semi-Autonomous, and Traditional Operating Assets and their interworking. Blockchain comes into play here for the automation of memorializing the custody chain.

In embodiments using an active, continuously-on Mote processor, we should expect little heat being required to maintain the minimum temperature for electron-exchange (i.e. the chemical-electrical transduction) if a heater envelopes the entire electronic assembly+battery and then is encased in a highly insulative material. Simply keeping the mistBee micro and it modest peripherals operating at longer duty cycles may provide enough heat generation to keep the battery chemistry ambient temperature above its point of electron valence exchange. More simply stated: When temperature declines, run the unit more often and consume more battery to then dissipate heat into an enveloping encapsulation material with a high heat distribution characteristic and make the inside of the mote “toasty” enough to keep the electrons flowing.

Example Magnetic Tags

Other embodiments may include use of magnetic tags, i.e., tags that are read from (and in some cases written to) using magnetic as opposed to RF fields. Example magnetic tags can for example provide two data packet read and write with a range of 5.5 meters (18 feet). Reading is accomplished for example with a air core electromagnet wound on a steel pipe having a thickness of 6.35 mm (0.25 inches).

Example Computer Vision Embodiments

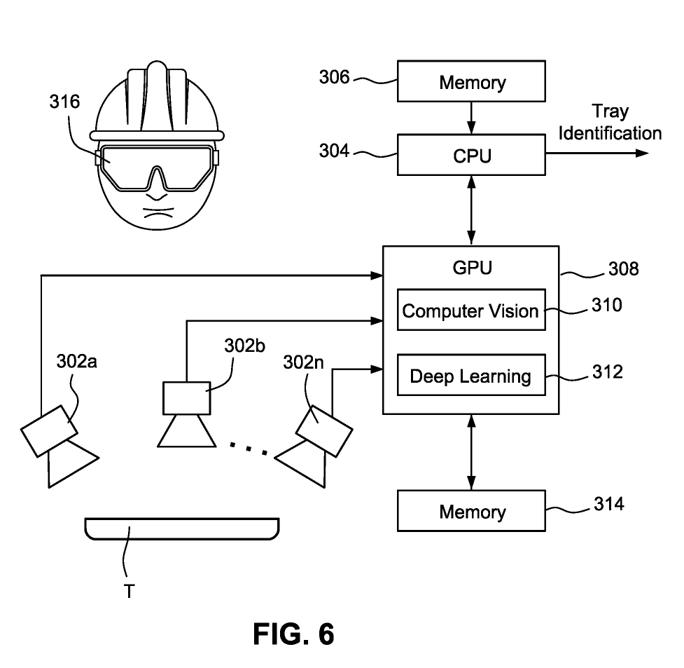

Instead of or in addition to the RFID tag technology described above, it is possible to use computer vision as shown in FIG. 6. Such embodiments can employ Computer Vision Systems for Virtual, Augmented, and Mixed Reality automated Surgical Tray population and Quality Assurance—both pre and post-Operation.

For example, one example embodiment has Humans teach Machines the proper composition of a Surgical Tray assembly, Machines to assist Humans in replicating the process, and Machines to teach other Machines to perform the Surgical Tray assembly.

In some example embodiments, a High-Resolution Camera technology is used to automatically harvest (i.e. characterize, raster-to-vector-to-3D-solid and the reverse) objects in the field of view and take instructions from the Human and provide direction back to the Human for orientation and placement of the object to capture all of its “machine readable” attributes.

Some embodiments enable optimized Nesting configurations of reconfigurable Surgical Trays, and provide instructions to Humans and Machines through Visual Field Augmentation (i.e. Mixed Reality) as to the required/desired Surgical Tray setup and instrument population.

Example embodiments provide surgical Tray setup and instrument population to be based on the Physician, Procedure, Patient, Products (e.g. Full Joint Replacement), Provider (i.e. the capabilities of the Healthcare Facility where the Procedure is to be conducted), and the Payer (i.e. what amount of Money is permitted to be spent).

In the example non-limiting FIG. 6 scanning system, the tray T is scanned by an optical scanning system 302 including plural cameras 302a, 302b, . . . 302n to provide multidimensional imaging of the tray and its contents. Other sensing technologies (e.g., RADAR, LIDAR, ultrasonics, acoustics, Xrays, or any desired sensors or combinations of sensors) may be used instead or in addition. In some example non-limiting embodiments, the sensors 302 could be part of an augmented reality or mixed reality system (e.g., mounted on or as part of glasses, goggles or the like) so that for example when an operator looks at the tray, the tray's content are automatically scanned and corresponding legends appear on the glasses/goggle view that identify each item in the tray and also indicate the quantity of each item in the kit (e.g., “Scalpel #21 Blade 1 of 3”, “Senn retractor 1 of 1”, “Blumenthal rongeurs 11 cm 1 of 1”, etc.)

In addition to identifying the type of instrument, the scanning system can be used to auto-identify the particular instrument (i.e., the particular instance of the instrument, e.g., this pair of rongeurs as opposed to that pair of seemingly identical rongeurs) based for example on graphical markings (e.g., graphical imagery, OCR, 1D/2D Barcode as DPM (Direct Part Marking) and separately applied labeling). In some embodiments, embedded or affixed active or passive transceivers/RFID transponders as discussed above can be used to provide or assist in providing such identification.

Once the tray T is scanned, the image stream(s) can be applied to a computer vision processor 310 of a graphics processing unit 310. In some cases, a deep learning accelerator 312 may be used to accelerate operation of a trained deep neural network used to recognize the items within the tray T as well as the tray itself. In other example non-limiting embodiments, visual-audial-tactical comparison may be made to a separate reference next-best-to-original digital facsimile. The computer vision 310 can automatically assimilate the 3-Dimensional geometry and physical attributes of an asset to provide Person-Product-Property association (e.g. surgical instruments, orthopedic implants, human musculoskeletal, surgical trays, sterilization containers) using camera 302 technology accompanied by other spatial (e.g. RADAR, LiDAR, Acoustics), and physical material sensing (e.g. weight, volume, chemical composition, mechanical composition, temperature & heat-related attributes, etc.)

Some embodiments may use mixed and/or augmented and/or virtual reality to train scrub technicians and to teach robots the assembly+population and depopulation+disassembly of surgical kits for specific surgical procedure as determined by a combination of patient+products+physician+provider+payer. Because the number of parts is far fewer, the size of the parts is much larger, and the precision of placing the parts in positions within trays has much larger tolerance/margin for error, what we are doing is much easier than what has been accomplished with respect to other industries' accomplishments. What is unique is the custom-build of the surgical kit based on patient+physician+products+procedure+provider location. Systems and processes perfected in manufacturing automation for automotive, computer, and micro-electronics contexts may be used to customize for the patient+physician+products+procedure+provider location specific surgical tray assembly/disassembly automation.

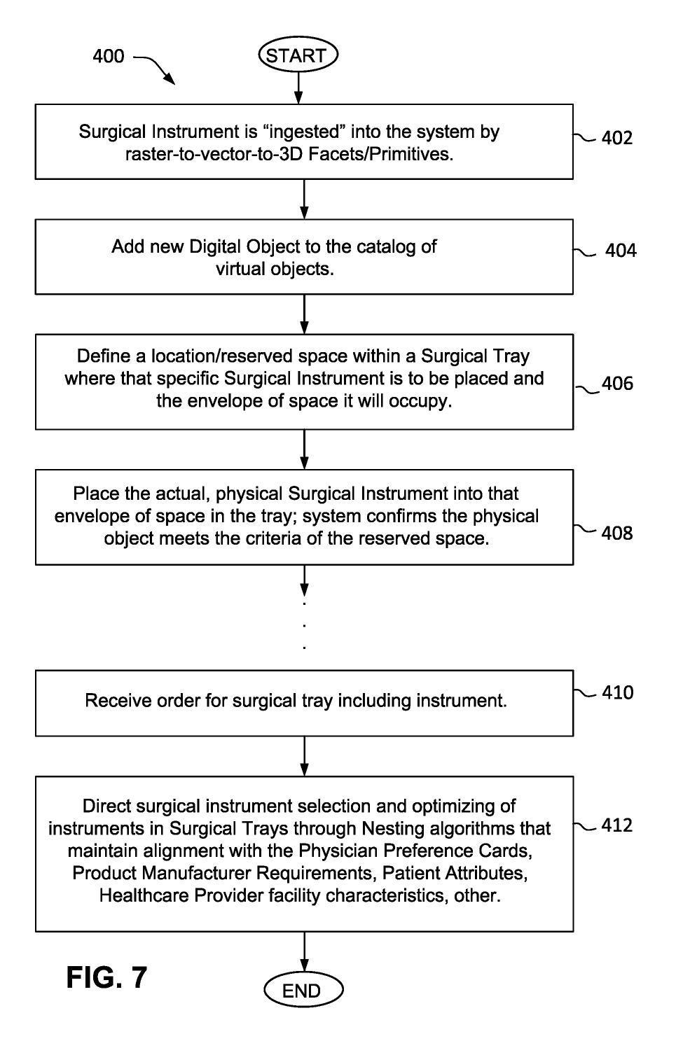

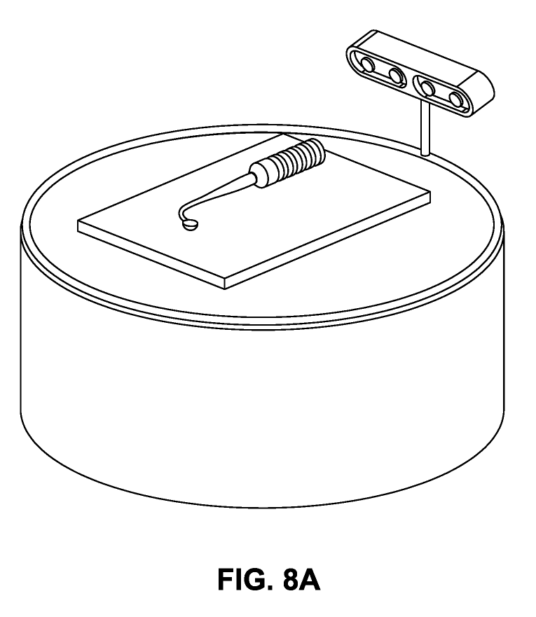

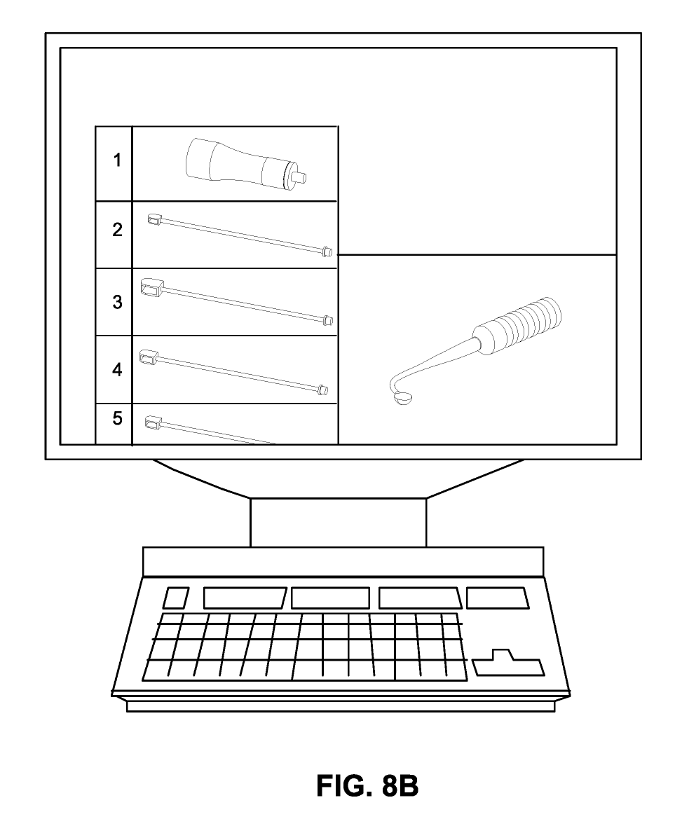

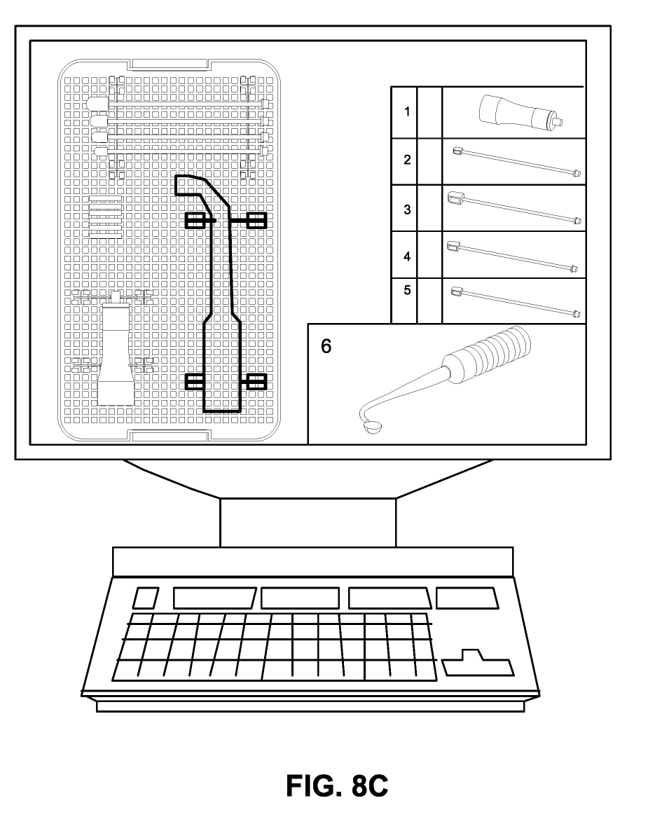

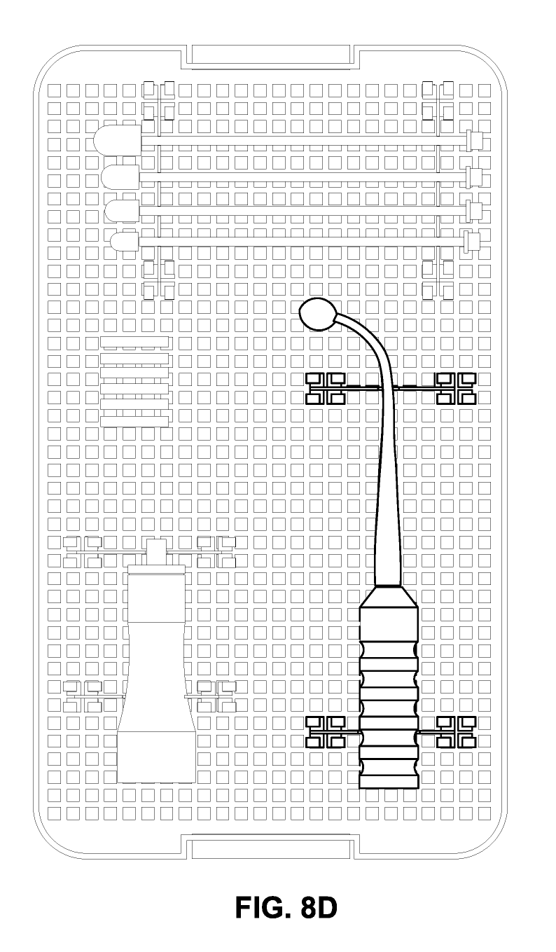

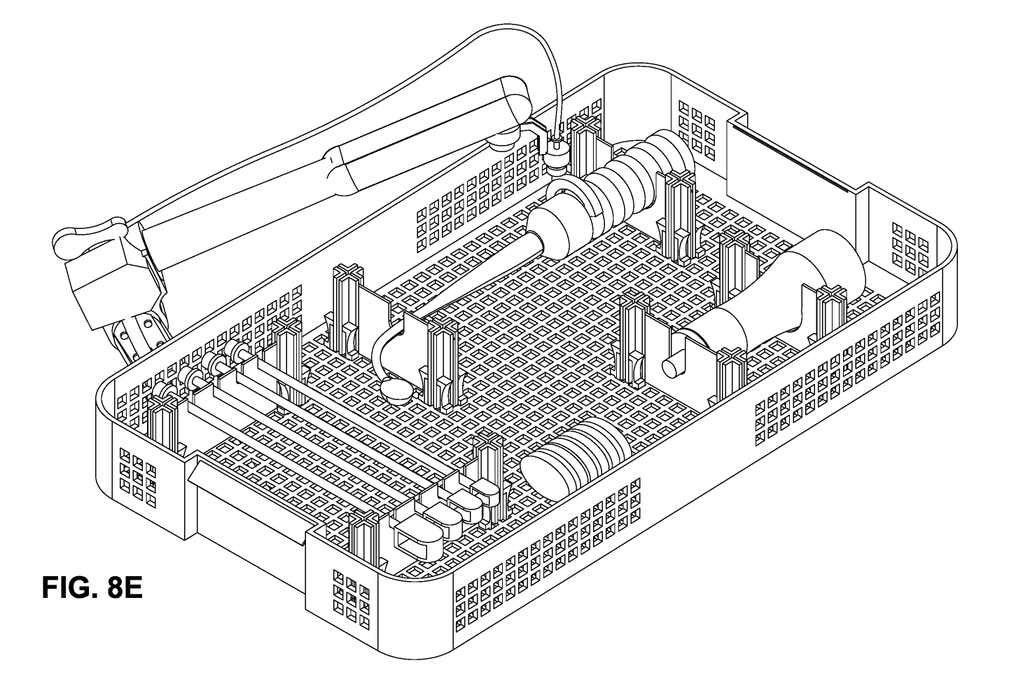

FIGS. 7 and 8 show an example non-limiting process. For context, the following are useful source materials:
an animation of a reconfigurable surgical tray offering at: https://www.klmedtech.com/
a video of a reconfigurable surgical tray offering of OneTray at: https://youtu.be/wCHSbs2_jv8
see also info at https://www.iststerilization.com/

A Mixed Reality Auto ID embodiment may perform or provide the following:

a. Surgical Instrument is “ingested” into the system by raster-to-vector-to-3D Facets/Primitives (block 402; FIG. 8A).

b. The addition of this new Digital Object to the catalog of virtual objects (block 404; FIG. 8B). In one example non-limiting embodiment/application, an original design database of the Asset(s) can serve to pre-populate the catalog/library of digital Assets. In other embodiments/applications, a physical example of the surgical instrument is scanned and this information is used to populate the catalog/library. Either or both techniques can be used depending on the particular instrument and other factors.

c. Depiction of a Surgical Tray containing a location where that specific Surgical Instrument is to be placed and the “highlighting” of the envelope of space it will occupy (block 406; FIG. 8C).

d. The placement of the actual, physical Surgical Instrument into that envelope of space with the “highlighting” going away when the system confirms that the physical object meets the criteria of the “highlighted” space (block 408; FIG. 8D).

e. Receipt of an order, instruction or script for a surgical kit comprising a surgical tray that may include this and other Surgical Instruments (block 410)

e. The directing of surgical instrument selection and optimizing of instruments in Surgical Trays through Nesting algorithms that maintain alignment with the Physician Preference Cards, Product Manufacturer Requirements, Patient Attributes, Healthcare Provider facility characteristics, et cetera. (block 412)

f. Population of a surgical tray based on the above, which may be performed automatically by a Fanuc or other robot. (FIG. 8E)

The building of reconfigurable surgical trays and the population of same with instruments may in some example non-limiting embodiments, be done through a HMI—Human-Machine Interface then serving to generate the CL—Centerline Gerber/Plot File data for CNC—Computer Numerical Control production (i.e. Additive and Subtractive Manufacture) and Human and/or Robotic Assembly of reconfigurable (or non-reconfigurable) Surgical Trays and the Human and/or Robotic population of Instruments in the Surgical Tray. The HMI may in some embodiments comprise an augmented, virtual or mixed reality HMI involving the use of goggles 316, display eyeglasses, or viewing through a window or screen such as a tablet screen. See e.g., Magic Leap Mixed Reality technology demonstration—a brief highlight depicts a Mixed Reality capability compatible with the technology herein: https://youtu.be/dX1x47UG6VI. Conventional marking and/or position detection technology can be used to reference viewer viewpoint to world space coordinates of a virtual space used to model surgical instruments and trays. Conventional graphics generation hardware such as graphics processing units may be used to display various views (including exploded views) of virtual surgical trays under construction.

The above approach is in some ways similar to the game Tetris® where alignment of a minimum number of consecutive color squares eliminates that row. Instead of being a fanciful game, the example non-limiting technology herein uses such an approach for the building and the recycling of Asset Assemblies in the Perioperative Lifecycle context, to help save lives.

The CL CNC and predicted Time and Motion data may be used for determining the end-to-end Instrument-Populated Surgical Tray assembly and disassembly to create Manufacturing/Process Routings as input to ERP—Enterprise Resource Planning-Scheduling-Execution systems.

Separately, overall Scheduling and Dispatch operations can be used to deliver Surgical Sets to points of use through traditional 3PL—3rd Party Logistics Warehousing Receiving-Put away-Picking including in some embodiments the use of Traditional Vehicle, Semi-Autonomous Vehicle, and fully Autonomous Delivery Vehicles (such as both Terrestrial and Aerial Drones) to have Surgical Trays delivered for completion of a Surgical Case Set (many Surgical Sets for Surgical Cases comprise multiple Surgical Trays) at the point of the Surgical Procedure or a point of interception in advance of the Surgical Procedure start. Such techniques may be thought of as a dynamic, on-the-fly, courier and cross-docking activity supporting the input to and output from the continuously running surgical suite.

Example Non-Limiting Features and Advantages Include:

Increased profitability and productivity
All in one—active asset tracking with integrated security platform
Small tags—better communication, reliability and accurate location communication

Asset Tracking
localization of medical devices in real time with a reading accuracy of 3-5 meters
enterprise solution offers central monitoring
easy data exchange between different building sections and hospitals
Increased patient safety and loss asset avoidance

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

For example, while non-limiting embodiments described above use radio frequency non-contact communication technology, other technologies such as the IEEE 1902.1 RuBee Magnetic ID technology are also possible.

All references above are hereby incorporated by reference for all purposes as if expressly set forth.

1. An article of manufacture comprising:
a conductive surface;
a galvanic connection to the conductive surface;
a transponder circuit operatively coupled to the galvanic connection; and
an elongated radio frequency radiator operatively coupled to the transponder circuit, the radio being suspended above the conductive surface to enable the conductive structure to operate as a ground plane for the radiator.

2. The article of claim 1 wherein the conductive surface comprises surgical steel.

3. The article of claim 1 further including an encapsulant that encapsulates the transponder circuit and radiator, the encapsulant providing a dielectric disposed between the conductive surface and the radiator.

4. The article of claim 3 wherein the encapsulant is impervious to the effects of an autoclave, does not carry pathogens, is able to be sterilized, and is substantially transparent to radio frequencies.

5. A method of manufacturing an article comprising:
electrically coupling an RFID die to a conductive surface of an article;
attaching an RF radiator to the RFID die; and
encapsulating the RFID die and the attached RF radiator in an encapsulant to provide sealing, binding and adherence of the RFID die and the RF radiator to the article.

6. The method of claim 5 wherein the article comprises a steel surgical instrument.

7. The method of claim 5 wherein the RFID die includes IEEE 802.15.4 wireless technology.

8. The method of claim 5 wherein the article operates as a ground plane for the RF radiator.

9. The method of claim 5 wherein the encapsulant is impervious to high temperatures of an autoclave.

10. A method of tracking assets comprising:
automatically reading an Advanced Shipping Notice (ASN), confirmed physical contents and condition at the point of the pre-trip loaded inventory, and a continuous monitoring of loaded inventory condition from a mote disposed in a container associated with surgical instruments or other assets; and
providing end-to-end chain of custody tracking of said container,
wherein the mote is configured to withstand an autoclave.

11. The method of claim 10 further including heating the mote by periodically operating it.

12. The method of claim 10 wherein the mote is disposed in or on surgical steel.

13. The method of claim 10 wherein automatically reading is performed at least one of optically, acoustically, magnetically and electromagnetically.

14. The method of claim 10 further including providing a virtual or augmented reality display indicating at least some of the information read from the mote.

15. The method of claim 10 wherein the reading is performed while the container is within an autoclave.

16. A process for manufacturing a surgical tray comprising:
(a) scanning a surgical instrument;
(b) in response to the scanning, adding the surgical instrument as a digital object to a catalog;
(c) using at least one computer, defining a space envelope within a surgical tray for placement of the surgical instrument;
(d) confirming the surgical instrument fits within the defined space envelope; and
(e) using the at least one computer, directing selection of said surgical instrument within other surgical instruments in a surgical tray through a nesting algorithm that maintains alignment with at least some physician preferences, surgical instrument manufacturer requirements, patient attributes and healthcare provider facility characteristics.
